(19) United States
(12) Patent Application Publication
(10) Pub. No.: US 2025/0288472 A1
(43) Pub. Date: Sep. 18, 2025

(54) PANT TYPE ABSORBENT ARTICLE

(71) Applicant: The Procter & Gamble Company, Cincinnati, OH (US)

(72) Inventors: Le KANG, Beijing (CN); Yin HU, Beijing (CN); Fancheng WANG, Beijing (CN)

(21) Appl. No.: 19/077,216

(22) Filed: Mar. 12, 2025

(30) Foreign Application Priority Data
Mar. 13, 2024 (WO) PCT/CN2024/081389

(51) Int. Cl. A61F 13/496; A61F 13/49

(52) U.S. Cl. CPC A61F 13/496; A61F 13/49011

(57) ABSTRACT

The present disclosure relates to a disposable absorbent article having a front region, a back region, a crotch region, and a main body including an absorbent core disposed on the crotch region. At least portion of the front and back regions are connected at first and second seams forming leg openings and a waist opening. The front region includes a front waist zone having a front waist elastic force factor, a front first zone having a front first zone elastic force factor, a front second zone having a front second zone elastic force factor, and a front third zone having a front third zone elastic force factor. The front second zone elastic force factor is lower than the front first zone elastic force factor and the front third zone elastic force factor, and the front first zone elastic force factor is higher than the front third zone elastic force factor.

### FIELD OF THE INVENTION

The present invention relates to disposable pull-on garments such as menstrual pants, disposable diapers and the like.

### BACKGROUND OF THE INVENTION

Pull-on wearable articles, or pant-type wearable articles, are those which are donned by inserting the wearer's legs into the leg openings and sliding the article up into position about the lower torso. They are intended to fit comfortably and snugly about the wearer. It is further desirable that they solve various kinds of skin troubles at side parts and/or upper leg parts during wearing the article.

Pull-on wearable articles having a main body to cover the crotch region of the wearer and a separate elastic belt defining the waist opening and leg opening are known in the art, such as described in PCT Publication WO 2006/17718A. Such pull-on wearable articles may be referred to as belt-type pants. On the other hand, certain pull-on wearable articles are configured such that the outer cover of the wearable main body completely covers the entirety of the garment-facing surface of the article. Such pull-on wearable articles may be referred to as unibody pants.

It may be desirable in some products to run elastics continuously across the belt in a transversal direction and/or along the entire periphery of a crotch area in order to ensure full elastic fit about the waist and leg. However, running elastics continuously across the belt and/or along the entire periphery of a crotch may create too much tension in force zones and/or elastic sections in the belt and/or crotch area causing uncomfortable tight feel.

Japanese patent 5933193B discloses a pants type disposable diaper with an exterior sheet comprising a stretchable nonwoven fabric and non-stretchable zone along leg periphery by applying a non-stretchable material. EP2572689 discloses a disposable diaper comprising a front and a rear waist regions respectively include first through third sub-regions which are adjacent one to another in this order, and a tensile stress of the first and third sub-regions is higher than that of the second sub-region.

There is still an unmet need for providing such a pant type absorbent article fit and comfort during wearing.

There is also a need for a disposable absorbent article to provide an undergarment-like look and feel.

### SUMMARY OF THE INVENTION

The present invention provides a disposable absorbent article having a longitudinal direction and a transversal direction perpendicular to the longitudinal direciton, and comprising a front region, a back region, a crotch region between the front region and the back region in the longitudinal direction, and a main body comprising an absorbent core disposed to cover continuously the crotch region and at least part of the front region and back region wherein the front and back regions are connected each other such that a first and a second seams are formed so that the absorbent article comprises leg openings and a waist opening; wherein the front region comprises, in order in the longitudinal direction, a front waist zone forming a waist edge and having a front waist clastic force factor, a front first zone having a front first zone elastic force factor, a front second zone having a front second zone elastic force factor, a front third zone having a front third zone clastic force factor, and a front fourth zone having a front fourth zone clastic force factor; wherein the front second zone includes a virtual transversal line bisecting the first and second side scams in a longitudinal direction; wherein the front second zone elastic force factor is lower than the front first zone elastic force factor and the front third elastic force factor; and wherein the front first zone clastic force factor is higher than the front third zone elastic force factor.

The present invention also provides a disposable absorbent article having a longitudinal direction and a transversal direction perpendicular to the longitudinal direciton, and comprising a front region, a back region, a crotch region between the front region and the back region in the longitudinal direction, and a main body comprising an absorbent core disposed to cover continuously the crotch region and at least part of the front region and back region, wherein the front and back regions are connected each other such that a first and a second seams are formed so that the absorbent article comprises leg openings and a waist opening; wherein the back region comprises, in order in the longitudinal direction, a back waist zone forming a waist edge and having a back waist elastic force factor, a back first zone having a back first zone clastic force factor, a back second zone having a back second zone clastic force factor, a back third zone having a back third zone clastic force factor, and a back fourth zone having a back fourth zone clastic force factor; wherein the back second zone includes a virtual transversal line bisecting the first and second side seams in a longitudinal direction; and wherein the back second zone clastic force factor is lower than the back first zone clastic force factor and the back third zone clastic force factor.

### BRIEF DESCRIPTION OF THE DRAWINGS

While the specification concludes with claims particularly pointing out and distinctly claiming the subject matter which is regarded as forming the present invention, it is believed that the invention will be better understood from the following description which is taken in conjunction with the accompanying drawings in which like designations are used to designate substantially identical elements, and in which:

FIG. 1 is a perspective view of an exemplary disposable pull-on garment of the present invention in a typical in-use configuration.

FIG. 2 is a perspective view of another exemplary disposable pull-on garment of the present invention in a typical in-use configuration.

FIG. 3 is a plan view of an exemplary disposable pull-on garment of the present invention in its flat uncontracted condition showing the wearer-facing surface.

FIG. 4 is a plan view of another exemplary disposable pull-on garment of the present invention in its flat uncontracted condition showing the wearer-facing surface.

FIG. 5 is a schematic cross section view taken along line 5-5 in FIG. 3 of an exemplary disposable pull-on garment.

FIG. 6 is a schematic cross section view taken along line 6-6 in FIG. 4 of an exemplary disposable pull-on garment.

FIG. 7 is a schematic view of an example of a sample holding fixture according to the Zone Tensile Force Measurement.

### DETAILED DESCRIPTION OF THE INVENTION

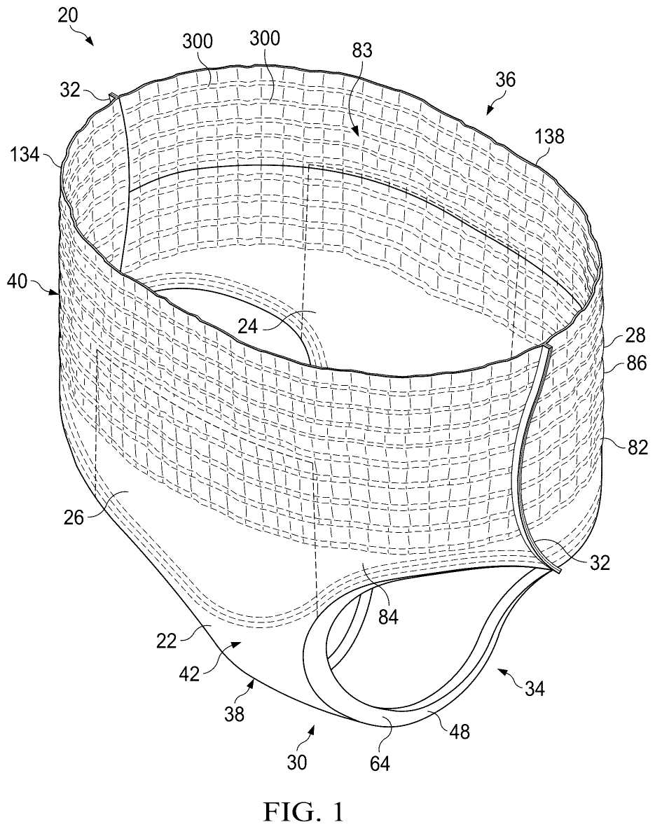

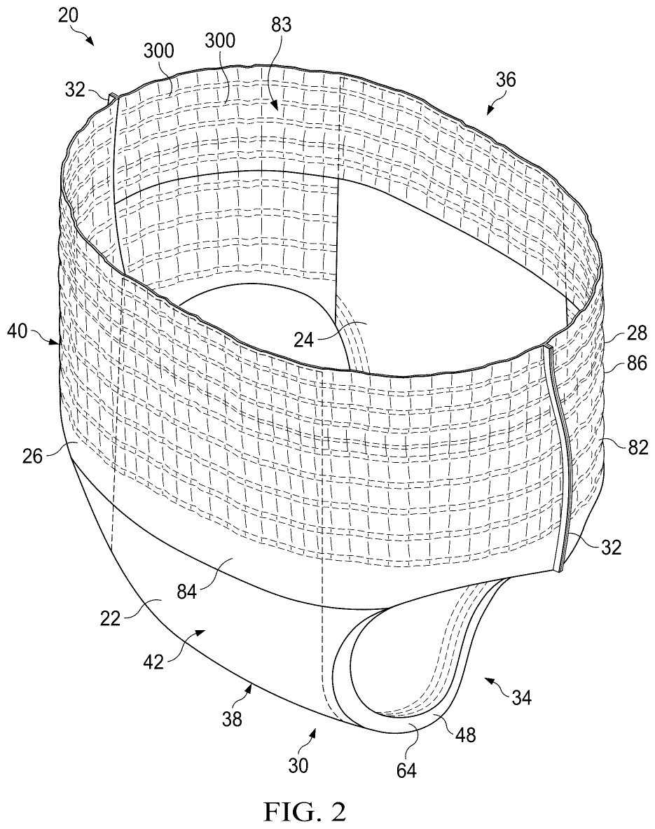

As used herein, the term “pull-on garment” refers to articles of wear which have a defined waist opening and a pair of leg openings and which are pulled onto the body of the wearer by inserting the legs into the leg openings and pulling the article up over the waist. The term “disposable” is used herein to describe garments which are not intended to be laundered or otherwise restored or reused as a garment (i.e., they are intended to be discarded after a single use and, preferably, to be recycled, composted or otherwise disposed of in an environmentally compatible manner). The pull-on garment is also preferably “absorbent” to absorb and contain the various exudates discharged from the body. Exemplary embodiments of the absorbent article are disposable absorbent pull-on garments, shown in FIGS. 1 and 2.

As used herein, the term “absorbent article” refers to pull-on garments generally worn by infants and other incontinent individuals to absorb and contain urine, feces and/or menses. It should be understood, however, that the term absorbent article is also applicable to other garments such as training pants, incontinent briefs, feminine hygiene garments or panties, and the like.

As used herein, the term “joined” encompasses configurations whereby an element is directly secured to another element by affixing the element directly to the other element, and configurations whereby an element is indirectly secured to another element by affixing the element to intermediate member(s) which in turn are affixed to the other element.

As used herein, a material or region having “no stretchability”, or a material or region is “no stretchable” refers to a material or region that, upon application of a biasing force, can stretch to an elongated length of no more than about 130% of its relaxed, original length (i.e. can stretch no more than 30 percent its original length), without rupture or breakage, and upon release of the applied force, shows little recovery, less than about 20% of its elongation without complete rupture or breakage as measured by EDANA method 20.2-89. In the event such an elongatable material recovers at least 40% of its elongation upon release of the applied force, the elongate-able material will be considered to be “elastic” or “elastomeric.” For example, an elastic material that has an initial length of 100 mm can extend at least to 150 mm, and upon removal of the force retracts to a length of at least 130 mm (i.e., exhibiting a 40% recovery). In the event the material recovers less than 40% of its elongation upon release of the applied force, the elongatable material will be considered to be “substantially non-elastic” or “substantially non-elastomeric”. For example, an elongatable material that has an initial length of 100 mm can extend at least to 150 mm, and upon removal of the force retracts to a length of at least 145 mm (i.e., exhibiting a 10% recovery).

As used herein, “force factor” in each zone is a tensile force (N) in the zone divided by the zone area (m²).

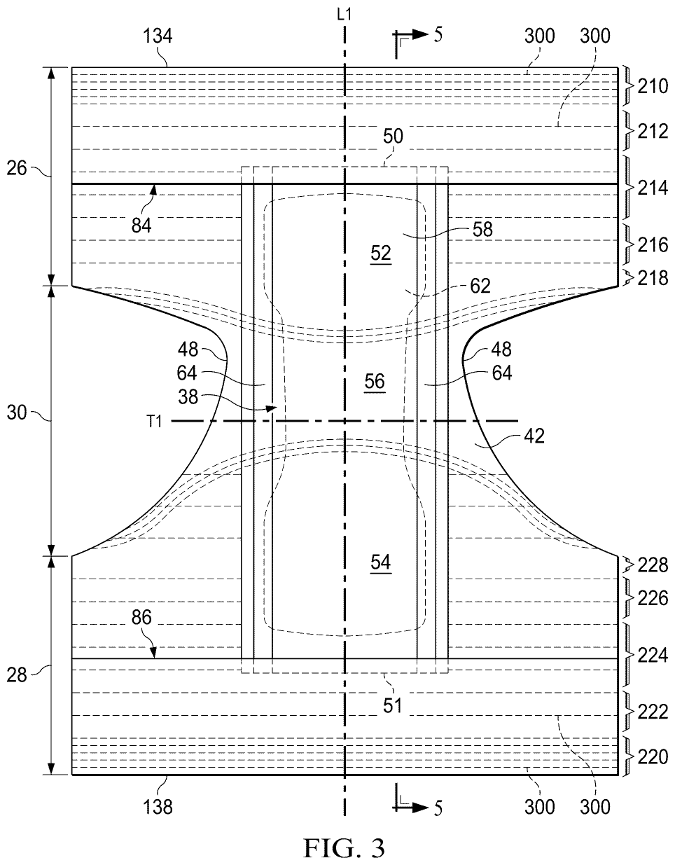

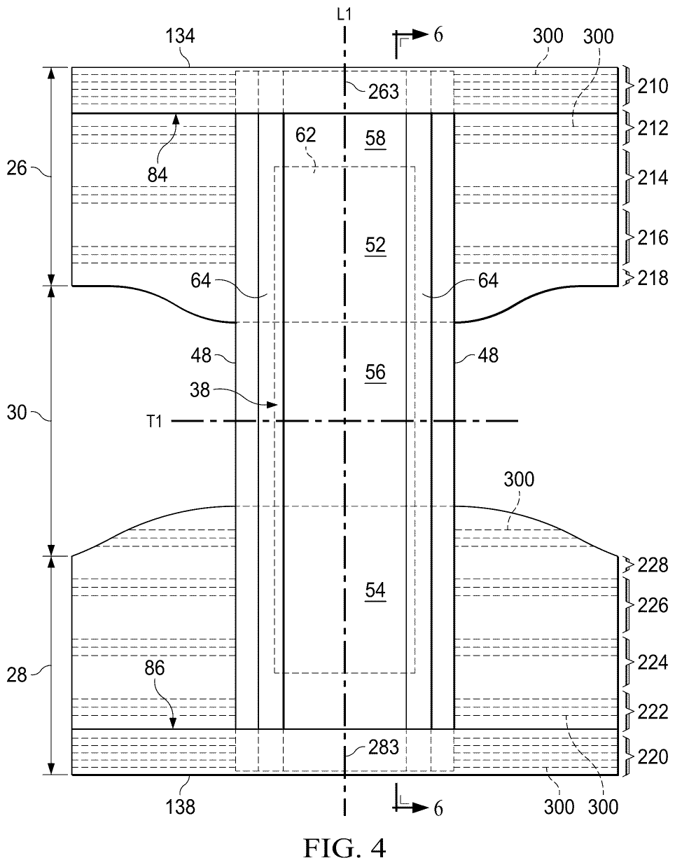

FIG. 1 is a perspective view of a unibody type disposable pull-on garment 20 disclosed herein. FIG. 2 is a perspective view of a belt type disposable pull-on garment 20 disclosed herein. Referring to FIGS. 3 and 4 as well, absorbent article 20 comprises an outer surface 22 which is a garment-facing surface, an inner surface 24 opposed to the outer surface 22, a front region 26, a back region 28 and a crotch region 30. The absorbent article 20 also comprises seams 32 which join the front region 26 and the back region 28 in their transverse edges to form two leg openings 34 and a waist opening 36. The absorbent article 20 has a longitudinal centerline L1 and a transverse centerline T1.

As shown in FIGS. 1 and 2, the absorbent article 20 comprises an absorbent main body 38 to cover the crotch region of the wearer and a belt 40 extending transversely about the waist opening 36. The absorbent article 20 may also comprise an outer cover layer 42 to cover the main body 38. The belt 40 defines the waist opening 36. The belt 40, the main body 38 and/or the outer cover layer 42 jointly define the leg opening 34. The absorbent article may comprise a leg elastic.

The main body 38 is disposed in the crotch region 30, and the front region 26 and back region 28 at least in part in a longitudinal direction so that the front, crotch and back regions are continuous. The front and back regions 26 and 28 may overlap at least a portion of the main body 38. The main body 38 comprises a left and a right longitudinally extending side edges 48 (hereinafter may be referred to as “longitudinal side edge”) and a front and a back transversely extending end edges 50, 51, respectively (hereinafter may be referred to as “front transverse end edge” and “back transverse end edge”, respectively). The main body 38 may be in a general rectangular shape, or be shaped such a way that side edges of the main body 38 are curved inwardly.

The main body 38 also has a front waist panel 52 positioned in the front region 26 of the absorbent article 20, a back waist panel 54 positioned in the back region 28, and a crotch panel 56 between the front and back waist panels 52, 54 in the crotch region 30.

The front transverse end edge 50 of the main body 38 may extend to the front region 26 and be spaced away from the front waist edge 134 as shown in FIG. 3, or may extend to the front waist edge 134 or extend close to the front waist edge 134 as shown in FIG. 4. The back transverse end edge 51 of the main body 38 may extend to the back region 28 and be spaced away from the back waist edge 138 as shown in FIG. 3, or may extend to the back waist edge 138 or extend close to the back waist edge 138 as shown in FIG. 4.

The main body 38 comprises a topsheet 58, a backsheet 60 and an absorbent core 62 disposed between the topsheet 58 and the backsheet 60 for absorbing and containing body exudates.

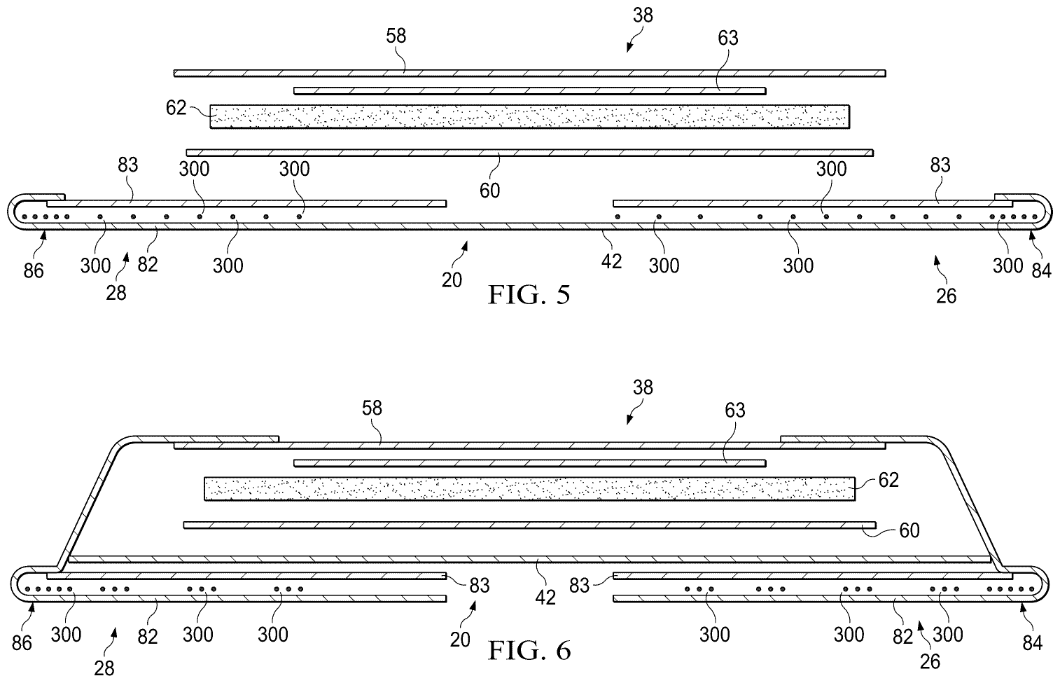

As shown in FIG. 5 showing an exemplary unibody type absorbent article, the absorbent articles 20 may comprise front and back belts 84, 86 disposed in the front and back regions 26, 28 respectively and intended to encircle at least a portion of the waist of the wearer, the front and back belts 84, 86 being connected by the main body 38 forming the crotch region 30 of the article 20. The front and back belts 84, 86 may be formed from a first belt layer 82 extending from a front waist edge 134 in a front region 26 through the crotch region 30 to a longitudinally opposing back waist edge 138 in a back region 28 and forming at least a portion of the outer surface of the absorbent article 20. The front and back belts 84, 86 also may comprise a second belt layer forming a portion of the inner surface 24 of the absorbent article.

The first belt layer 82 may form a portion of the outer surface 22. In the unibody type product, the first belt layer 82 may extend from a front waist edge 134 in a front region 26 through the crotch region 30 to a longitudinally opposing back waist edge 138 in a back region 28 and form a portion or the whole of the outer surface 22 of the absorbent article 20. In one embodiment, referring to FIGS. 3 and 5, the outer cover layer 42 may extend from a front waist edge 134 in a front region 26 through a crotch region 30 to a longitudinally opposing back waist edge 138 in a back region 28 and forming a portion of or the whole of the garment-facing surface 22 of the absorbent article 20. In such a case, the outer cover layer 42 forms the first layer 82.

The second belt layer may overlap at least a portion of the main body and form the outer surface of the first belt layer or alternatively the inner surface of the first belt layer. Alternatively, the inner surface and outer surface of each of the front and back belts is formed from a single web of material. As shown in FIG. 5, the first belt layer 82 may form a portion of the garment facing surface, may fold over and be connected to the second belt layer 83, such that the first belt layer also forms a portion of the wearer facing surface.

As shown in FIG. 6 showing an exemplary belt type absorbent article, the absorbent articles 20 may comprise front and back belts 84, 86 intended to encircle at least a portion of the waist of the wearer, the front and back belts 84, 86 being connected by a main body 38 forming the crotch region 30 of the absorbent article 20. The front and back belts 84 and 86 may be formed from a first belt layer forming a portion of the outer surface 22 of the absorbent article, the first belt layer 82 (also referred to as an outer belt layer) may be formed of two longitudinally spaced webs of material. The front and back belts 84 and 86 may also comprise a second belt layer (also referred to as an inner belt layer) 83 forming a portion of the inner surface 24 of the absorbent article 20. The first and second belt layers 82, 83 may be formed of substantially the same material or may comprise different materials. The first and second belt layers 82, 83 may be formed from nonwovens, films, foams, elastic nonwoven, or combinations thereof. In a belt type absorbent article, the first belt layer may comprise a material different from the outer cover layer.

The front and back belts 84, 86 may also comprise an elastomeric material disposed between the first and second belt layers 82, 83. The elastomeric material may comprise one or more elastic strands, elastomeric films (including apertured films), elastomeric ribbons, elastomeric nonwovens, elastomeric filaments, elastomeric adhesives, elastomeric foams, scrims or combinations thereof. A portion of the elastomeric material may be directly combined with the optional outer cover layer.

In one embodiment, the elastomeric material comprises a plurality of elastics.

Each of the elastics in the front and back belts, as well as the crotch region, may have the same dtex, Dt, and the same pre-strain, PS. Another suitable embodiment may have each of the elastic materials of the front and back belts have the same dtex, and different pre-strain about 180 to about 300% strain with lower dtex (smaller diameter of strands) and/or lower pre-strain (less pre-strain) in an area in the crotch region. The front and back belt 84, 86 may comprise any known materials. Suitable material for the front and back belt 84, 86 can be manufactured from a wide range of materials such as plastic films; apertured plastic films; woven or nonwoven webs of natural materials (e.g., wood or cotton fibers); synthetic fibers (e.g., polyolefins, polyamides, polyester, polyethylene, or polypropylene fibers), or a combination of natural and/or synthetic fibers; or coated woven or nonwoven webs. The belt may comprise a nonwoven web of synthetic fibers. The belt may comprise an inner hydrophobic, non-stretchable nonwoven material and an outer hydrophobic, non-stretchable nonwoven material.

The front and back belts 84, 86 may overlap at least a portion of the main body and one or both of the belt portions may be disposed on the outer surface of the main body or alternatively on the inner surface of the main body. A portion of the second belt layer and/or a portion of the first belt layer may be directly attached to the outer cover layer. Alternatively, at least a portion of the inner surface and outer surface of each of the belt portions may be formed from a single web of material. As shown in FIG. 6, the first belt layer 82 may form a portion of the garment facing surface, may fold over and be connected to the second belt layer 83, and may also be connected to a portion of the topsheet 58, such that the first belt layer 82 also forms a portion of the wearer facing surface.

The front region 26 may comprise a first side panel and a second side panel and a center panel sandwiched the first and the second side panels in the transversal direction. The back region 28 may comprise a first side panel and a second side panel and a center panel sandwiched the first and the second side panels in the transversal direction.

The front and back regions 26, 28 may overlap at least a portion of the main body 38, and one or both of the regions may be disposed on the outer surface of the main body or alternatively on the inner surface of the main body.

The front region and back region 26 and 28 may be formed from a first belt layer 82 forming a portion of the outer surface 22 of the absorbent article.

The front and back regions 26 and 28 may also comprise a second belt layer (also referred to as an inner belt layer) forming a portion of the inner surface 24 of the absorbent article 20.

Each of the front region and back region 26 and 28 comprises at least one material continuous along the transversal direction of the absorbent article. In one embodiment, the first belt layer in the front region 26 and/or the back region 28 is continuous along the transversal direction of the absorbent article.

The main body 38 of the absorbent article comprises backsheet 60, topsheet 58, and an absorbent core 62 disposed between the topsheet 58 and the backsheet 60. The backsheet may be formed of a nonwoven material, woven material, films or laminates comprising a combination of one or more of these materials. In one embodiment the backsheet is a film and nonwoven laminate wherein the nonwoven of the laminate is the outer cover layer.

The absorbent article may comprise at least one leg elastic that run parallel to, and a few millimeters inboard of, at least a portion of the leg cut outs. The leg elastic material may be disposed so as to extend generally longitudinally along the longitudinal side edge 48 of the main body 38. The leg elastic material may be disposed at least in the crotch region 30 of the absorbent article 20 or may be disposed along the entirety of the longitudinal side edge 48.

The main body 38 may additionally comprise elasticized barrier leg cuffs 64 disposed at or adjacent the longitudinal side edges 48 of the main body 38. The barrier leg cuff 64 provides improved containment of liquids and other body exudates in the crotch region 30. The barrier leg cuff 64 may comprise a single layer of material which may be folded to form a barrier leg cuff having two layers. The barrier leg cuff 64 extends from the side of the main body at or adjacent the longitudinal side edge 48 toward the longitudinal centerline L1. The barrier leg cuff may be folded back toward the longitudinal side edge 48.

The liquid pervious topsheet 58 may be positioned adjacent the body-facing surface of the absorbent core 62 and may be joined thereto and/or to the backsheet 60 by any attachment means known in the art. The liquid impervious backsheet 60 is generally that portion of the absorbent article 20 positioned adjacent the garment-facing surface of the absorbent core 62 and prevents the exudates absorbed and contained therein from soiling articles that may contact the absorbent article 20. The absorbent core is positioned between the topsheet 58 and the backsheet 60 and absorbs and retains liquids such as urine and other certain body exudates.

The topsheet 58, the backsheet 60 and the absorbent core 62 may be manufactured known materials. Suitable topsheet materials may include porous foams; reticulated foams; apertured plastic films; or woven or nonwoven webs of natural fibers (e.g., wood or cotton fibers), synthetic fibers (e.g., polyester or polypropylene fibers), or a combination of natural and synthetic fibers. Suitable backsheet materials may include breathable materials that permit vapors to escape from the diaper while still preventing exudates from passing through the backsheet.

A suitable absorbent core for use in the absorbent article 20 may comprise any absorbent material which is generally compressible, conformable, non-irritating to the wearer's skin, and capable of absorbing and retaining liquids such as urine and other certain body exudates. In addition, the configuration and construction of the absorbent core may also be varied (e.g., the absorbent core(s) or other absorbent structure(s) may have varying caliper zones, hydrophilic gradient(s), a superabsorbent gradient(s), or lower average density and lower average basis weight acquisition zones; or may comprise one or more layers or structures). In some embodiments, the absorbent core may comprise a fluid acquisition component, a fluid distribution component, and a fluid storage component.

Suitable absorbent cores of the present disclosure may comprise cellulosic airfelt material. For instance, such absorbent cores may comprise less than about 95%, 90%, 80%, 70%, 60, 40%, 50%, 40%, 30%, 20%, 10%, 5%, or even 1% of the cellulosic airfelt material as determined by weight. Additionally, such an absorbent core may be primarily comprised of an absorbent gelling material in amounts of at least about 5%, 10%, 20%, 30%, 40%, 50%, 60%, 70%, 80%, 85%, 90%, 95%, or even about 100% as determined by weight. Furthermore, a portion of the absorbent core may comprise a microfiber glue.

The outer cover layer 42 may be disposed on the outer surface 22 of the absorbent article 20 and may cover the crotch panel 56 of the absorbent main body 38. The outer cover layer 42 may extend into and cover the front waist panel 52 and the back waist panel 54 of the main body 38. The outer cover layer may form a portion of the backsheet and/or the main body. The outer cover layer 42 may be directly joined to and cover a portion or all of the liquid impervious backsheet 60 of the main body 38. Thus, the outer cover layer 42 may be disposed between the front and back belt 84, 86 and the liquid impervious backsheet 60 of the main body 38. In one embodiment, the outer cover layer 42 may be coextensive with the liquid impervious backsheet 60.

The outer cover layer 42 may comprise a material separate from the material of the inner belt layer 83 and/or the outer belt layer 82 constituting the belt 40. The outer cover layer 42 may comprise two or more layers of materials. The outer cover layer 42 may comprise any known materials and may comprise materials used for the front and back belt 84, 86 as explained above. The outer cover layer 42 may comprise a single layer of nonwoven web of synthetic fibers. The outer cover layer 42 may comprise a single layer of hydrophobic, non-stretchable nonwoven material. The outer cover layer may comprise a film, a foam, a nonwoven, a woven material or the like and/or combinations thereof such as a laminate of a film and a nonwoven.

The front belt 84 and the back belt 86 are permanently or refastenably connected at the seams 32 to form a belt 40 having a ring-like configuration.

Front region 26 may be disposed on the front belt 84. Referring to FIGS. 3 and 4, the front region 26 may comprise, in order in the longitudinal direction, a front waist zone 210 forming a waist edge 134 and having a front waist elastic force factor, a front first zone 212 having a front first zone elastic force factor, a front second zone 214 having a front second zone elastic force factor, a front third zone 216 having a front third zone clastic force factor, and a front fourth zone 218 having a front fourth zone clastic force factor. In the front region 26, the front second zone 214 includes a virtual transversal line bisecting the first and second side seams 32 in a longitudinal direction, the front second zone elastic force factor is lower than the front first zone elastic force factor and the front third zone elastic force factor. Further the front first zone elastic force factor is higher than the front third zone elastic force factor. The virtual transversal line bisecting the first and second side seams in a longitudinal direction may be parallel to transverse centerline T1. The virtual transversal line bisecting the first and second side seams in a longitudinal direction may not be completely parallel to transverse centerline T1 but slightly tilted about transversal centerline T1.

With the feature of a front second zone having lower elastic force than a front first zone and covering a tummy area, little pressure is given to the tummy area, so that the wearer feels more comfortable while wearer's wearing the absorbent article of the present invention.

With the feature of a front first zone having higher elastic force than a front third zone, the absorbent article of the present invention may provide a better fit as well as comfort to a wearer during wearing the absorbent article.

Higher elastic force of the upper front region 212 than that of the intermediate front region 214 may provide desirable fit yet intend to make it more comfortable to wear given the body dimension corresponding to the intermediate front region is larger than the body dimension corresponding to the upper front region 212.

The front second zone may have a length of about 30% to about 65%, 35% to about 60%, or 30% to about 50% of the length of the first scam.

The front waist zone and the front first zone may have a length in total in a longitudinal direction of about 15% to about 35% of the length of the first seam. The front waist zone and the front first zone may have a length in total in a longitudinal direction of about 20 mm to about 60 mm. If the total length of the front waist zone and the front first zone is too long, the absorbent article may not provide desirable comfort to the wearer during the absorbent article.

The front third zone may have a length of about 5% to about 30% of the length of the first seam.

In one embodiment, the front third zone elastic force may higher than the front fourth zone clastic force. The front third zone may have a length of about 5% to about 30% of the length of the first seam. In the embodiment, the back fourth zone may have no stretchability.

The front fourth zone may have a length of about 5% to about 9% of the length of the first scam.

The combination of having features that the front second zone elastic force factor is lower than the front first zone elastic force factor and the front third zone elastic force factor, and the front first zone clastic force factor is higher than the front third zone elastic force factor, and positioning the front second zone to include a virtual transversal line bisecting the first and second side seams 32 in a longitudinal direction, the absorbent article of the present invention comprising a front regions described herein can be desirably and stably fit around the wearer's body yet provides comfort feel, and mitigate sagging during wearing.

Back region 28 may be disposed on the back belt 84. Referring to FIGS. 3 and 4, the back region 28 may comprise, in order in the longitudinal direction, a back waist zone 220 forming a waist edge 134 and having a back waist elastic force factor, a back first zone 222 having a back first zone elastic force factor, a back second zone 224 having a back second zone elastic force factor, a back third zone 226 having a back third zone clastic force factor, and a back fourth zone 228 having a back fourth zone elastic force factor. In the back region 28, the back second zone 224 includes a virtual transversal line bisecting the first and second side seams 32 in a longitudinal direction, and the back second zone elastic force factor is lower than the back first zone clastic force factor and the back third zone elastic force factor.

With the back second zone having lower elastic force than the back first zone and covering a hip area, little pressure is given to the hip area which while a wearer's wearing the absorbent article of the present invention.

The back first zone elastic force may be higher than the back third zone elastic force.

The back second zone may have a length of about 30% to about 65% of the length of the first scam.

The back waist zone and the back first zone may have a length in total in a longitudinal direction about of about 5% to about 35% of the length of the first scam.

In one embodiment, the back third zone elastic force may higher than the back fourth zone clastic force. In the embodiment, the back fourth zone may have no stretchability. The back fourth zone may have a length of about 5% to about 9% of the length of the first seam. The fourth zone having no stretchability in the front region and/or the back region may reduce the potential uncomfortableness around the legs due to elastic force of these regions in conventional products.

Zones in the front region and the back region each may have the same elastic material, and/or clastic material arrangement. In one embodiment, the first zone 212, 222, the second zone 214, 224 and the third zone 216, 226 are not substantially different in clastic material and elastic material arrangement. Alternatively, the first zone 212, 222, the second zone 214, 224 and the third zone 216, 226 are different in clastic material and/or elastic material arrangement.

In either or both of front region 26 and back region 28, in any of the first, the second and the third zone, the side regions and the central region may differ in the elastic material or elastic material arrangement or elastic force. In one embodiment, in the elastic force of a central region is lower than the elastic force of the side regions. In the embodiment, the central region may have no stretchability. With such feature of side regions having elastic force and a central region having lower elastic force or substantially no stretchability, the absorbent article can provide desirable fit and comfort as the body where the side regions are placed has a smaller curvature than the body where the central region is placed.

Elastic materials in the front region 26 and/or the back region 28 may be disposed continuously along the entire front region 26 and/or back region 28 in a transversal direction, or transversally interrupted by the main body 38.

When any of the zones of the front region 26 and/or the back region 28 overlaps the main body 38, elastic materials in the front region 26 and/or the back region 28 may have one or more cuts in the area where the zone of the front region 26 and/or the back region 28 overlaps the main body 38.

The elastic strands may be distributed amongst different regions in the front and/or the back regions. Elastic strands may be distributed evenly amongst the different regions in the front and/or the back regions. Elastic strands may also be distributed unevenly amongst different regions in the front and/or the back regions.

The elastic strands 300 may have a linear density between 200 to 2500. Linear density is the density of the elastic fibers in the elastic strand. The most commonly used unit for the linear density is the decitex, abbreviated dtex, which is the mass in grams per 10,000 meters (Dt). The linear density may be used to change the force profile. For example, one could reach a desired force profile by selecting the linear density of a single elastic strand, combining multiple elastic strands with a smaller linear density in close proximity to each other, and/or combining with other elastomeric materials.

The number of elastic strands in each zone may be changed according to the placement of the absorbent core. The force profile must be adjusted depending on the location and thickness of the absorbent core.

The gaps between the elastic strands 300 may be 2 mm, 3 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, or 22 mm, or 25 mm.

The absorbent article 20 may have a graphic disposed in the front region 26 and/or the back region 28. Graphics may be printed on the outermost nonwovens (e.g. the outer belt layer 82, or the outer cover layer 42), either on the garment facing side or the body facing side of these nonwovens. Graphics may also be printed on the garment facing side of the inner belt layer 83. Graphics of the present disclosure may be printed in part on the backsheet and in part on the belt layer.

### Test Methods

These tests can be used for stretch laminates that comprise elastic strands, e.g. Spandex strands. Typically, the elastic strands are stretched and laminated between two nonwovens to make the stretch laminate. Since these tests will be done for each elastic zone, it represents a total value for all the elastics in that zone. These tests are suitable for pant type absorbent articles, comprising parallel strand elastics that run predominantly in the transverse direction of the product.

### 1. Elastic Force

### 1) Zone Tensile Force Measurement

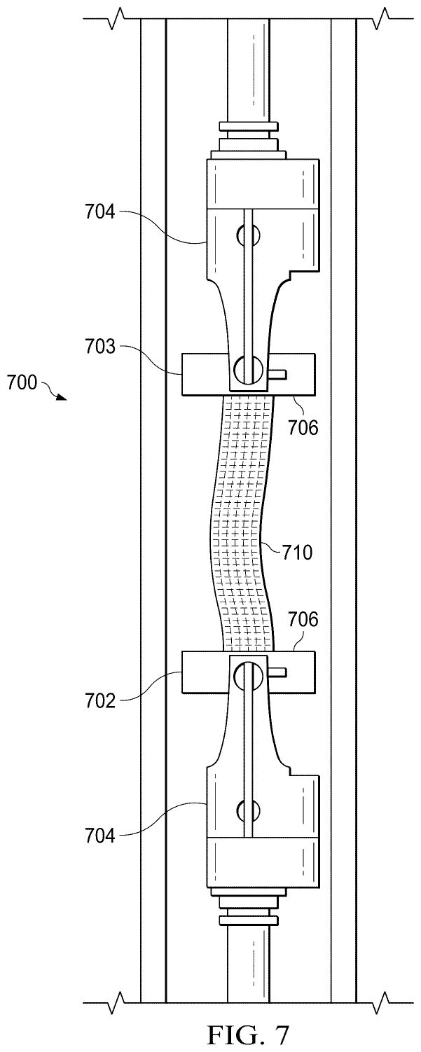

Force may be measured using an Electronic Tensile Tester with a computer interface such as the MTS Criterion C42 running TestWorks 4 Software (available from MTS SYSTEMS (CHINA) CO., LTD) or equivalent instrument. A load cell is chosen so that force results for the samples tested will be between 10 and 90% of capacity of the load cell. The instrument is calibrated according to the manufacturer's instructions. All testing is performed in a room maintained at 23°+2° C. and 50±5% relative humidity. The tensile tester is fitted with grip-type sample holding fixtures 700 as shown in FIG. 7. Each fixture comprises single line contact clamp 702 having a width at least as wide as a width of a test specimen 704.

To obtain a test specimen of each zone, the sample product is open along both side seams 32, and the front and back elastic belts 84, 86 are removed from the main body 38 by separating the bonding between the belts and main body. When the product is a unibody type pants product, a main body 38 is removed from remaining part of the product, and cut through the remaining part in a transversal direction to separate a front belt 84 and a back belt 86. Cold Spray may be used with paying attention not to make wrinkles in the belt sections. Care is taken not to spray on any belt elastics 300. The obtained front belt section and back belt section are severed into each zone in a rectangle shape of 210 (220), 212 (222), 214 (224), 216 (226) and 218 (228) according to the present invention to obtain a specimen of each zone. Each zone should be cut down separately in horizontal direction into a rectangle shape with care not to cut any elastic 300. Specimens are pre-conditioned at 23° C.±2° C. and 50%+5% relative humidity for two hours prior to testing.

The instrument is set up to go through the following steps.

| Crosshead Speed | 254.0 | mm/min |
|---|---|---|
| Data Acquisition Rate | 50 | Hz |
| Final Gauge Length | 0.5 × Full Stretch Circumference | |
| Hold Time | 3 | s |

| | |
|---|---|
| Number of Cycles | 1 |

Full Stretch Circumference of a product is measured by stretching the entire belt of the product by two hands: Insert two hands inside of the product in such a way that each hand is placed along each side seam, then fully stretch the belt using hands until the belt does not stretch any more. Measure the whole belt circumference length using a rubber ruler which does not include two side seams, and report it to the nearest 1 mm as Full Stretch Circumference.

Referring to FIG. 7, one end of the specimen 710 is clamped into the upper clamp 702 and the load is tared. The other end of the specimen 710 is clamped into the lower clamp 703. Approximately 5 mm-10 mm of each end of the specimen is behind the contact line 706 of the clamp 702, 703 so that the entire seam width is behind the contact line 706 of the clamp 702, 703. The crosshead 704 connected to the upper clamp 702 is raised slowly while the specimen is held in place by hand as necessary until the force is between 0.05N and 0.1N with care not to add any unnecessary pre-stretch force on the specimen. The gauge length at this point is the Initial Gauge Length.

The test is started and the specimen is extended to the half of the belt Full Stretch Circumference which is the Final Gauge Length at a crosshead speed of 254 mm/min, held for 3s and returned to the Initial Gauge Length at the same speed. The specimen is extended in the article transverse direction during the test. The unload force at 70% of the Final Gauge Length during the unload segments of the test is recorded.

Five articles are analyzed and the unload forces are recorded for each of the front and back zones 210 (220), 212 (222), 214 (224), 216 (226) and 218 (228). The average tensile force (N) is calculated to the nearest 0.01 N for each zone including the front and back specimens for that zone.

2) Zone Tensile Force Factor The tensile force factor (N/m²) in each zone is calculated by dividing a tensile force (N) in the zone by the zone area (m²) and reported to the nearest 0.1 N/m².

A zone area is calculated by multiplying a zone width and a zone length. A zone width is a width of the zone in a product transversal direction excluding a side seam width in each side. A zone length is a length of the zone along a seam.

### EXAMPLES

### Example 1

Unibody type menstrual pants were produced according to descriptions in Table 1 below.

TABLE 1

| | 1 | | 2 | | 3 | |
|---|---|---|---|---|---|---|
| | Front | Back | Front | Back | Front | Back |
| Product | region | region | region | region | region | region |
| Zone width (mm) | 570 | 570 | 570 | 570 | 570 | 570 |
| Waist zone | 50 | 50 | 20 | 20 | 20 | 20 |
| length (mm) | | | | | | |
| First zone | 10 | 10 | 10 | 10 | 31 | 31 |
| length (mm) | | | | | | |
| Second zone | 50 | 90 | 80 | 80 | 31 | 31 |
| length (mm) | | | | | | |
| Third zone | 50 | 10 | 10 | 10 | 31 | 31 |
| length (mm) | | | | | | |
| Fourth zone | 10 | 10 | 10 | 10 | 17 | 17 |
| length (mm) | | | | | | |
| Waist zone | 12, 620d, | 12, 620d, | 6, 620d, | 6, 620d, | 6, 620d, | 6, 620d, |
| elastics*¹ | 260% | 260% | 260% | 260% | 260% | 260% |
| First zone | 2, 620d, | 2, 620d, | 2, 620d, | 2, 620d, | 3, 620d, | 3, 620d, |
| elastics*¹ | 220% | 220% | 260% | 260% | 240% | 240% |
| Second zone | 5, 470d, | 5, 470d, | 5, 470d, | 5, 470d, | 3, 470d, | 3, 470d, |
| elastics*¹ | 220% | 220% | 220% | 220% | 220% | 220% |
| Third zone | 5, 620d, | 2, 470d, | 2, 620d, | 2, 620d, | 3, 620d, | 3, 620d, |
| elastics*¹ | 220% | 220% | 200% | 200% | 200% | 200% |
| Fourth zone | 0 | 0 | 0 | 0 | 0 | 0 |
| elastics*¹ | | | | | | |
| Waist zone elastic | 88.4 | 88.4 | 110 | 110 | 110 | 110 |
| force factor (N/m²) | | | | | | |
| First zone elastic | 59.6 | 59.6 | 73.7 | 73.7 | 32.6 | 32.6 |
| force factor (N/m²) | | | | | | |
| Second zone elastic | 24.6 | 13.6 | 15.4 | 15.4 | 23.5 | 23.5 |
| force (N/m²) | | | | | | |
| Third zone elastic | 29.8 | 49.1 | 62.0 | 62.0 | 26.5 | 26.5 |
| force factor (N/m²) | | | | | | |
| Fourth zone elastic | 0 | 0 | 0 | 0 | 0 | 0 |
| force (N/m²) | | | | | | |

| | 4 | | 5 | |
|---|---|---|---|---|
| | Front | Back | Front | Back |
| Product | region | region | region | region |
| Zone width (mm) | 570 | 570 | 570 | 570 |
| Waist zone | 20 | 20 | 20 | 20 |
| length (mm) | | | | |
| First zone | 22 | 22 | 25 | 25 |
| length (mm) | | | | |
| Second zone | 52 | 52 | 37.5 | 37.5 |
| length (mm) | | | | |
| Third zone | 32 | 42 | 25 | 25 |
| length (mm) | | | | |
| Fourth zone | 4 | 4 | 10 | 10 |
| length (mm) | | | | |
| Waist zone | 6, 620d, | 6, 620d, | 6, 620d, | 6, 620d, |
| elastics*¹ | 260% | 260% | 280% | 280% |
| First zone | 3, 620d, | 3, 620d, | 2, 620d, | 2, 620d, |
| elastics*¹ | 220% | 220% | 260% | 260% |
| Second zone | 3, 620d, | 3, 620d, | 3, 620d, | 3, 620d, |
| elastics*¹ | 220% | 220% | 260% | 260% |
| Third zone | 3, 620d, | 3, 620d, | 2, 620d, | 2, 620d, |
| elastics*¹ | 220% | 220% | 240% | 240% |
| Fourth zone | 0 | 0 | 0 | 0 |
| elastics*¹ | | | | |
| Waist zone elastic | 109 | 109 | 121 | 121 |
| force (N/m²) | | | | |
| First zone elastic | 41.1 | 41.1 | 29.5 | 29.5 |
| force (N/m²) | | | | |
| Second zone elastic | 18.0 | 18.0 | 29.5 | 29.5 |
| force (N/m²) | | | | |
| Third zone elastic | 36.0 | 36.0 | 26.7 | 26.7 |
| force (N/m²) | | | | |
| Fourth zone elastic | 0 | 0 | 0 | 0 |
| force (N/m²) | | | | |

*¹numbers of elastics, dtex of elastic, pre-stretch of elastic

### Example 2

Chinese consumers (n=5) were asked to wear an invention product (Product 2) and a comparison product (Product 5) for more than 1 hour during day, respectively, with movement including walking, squat and sit. The consumers were then asked to give comments on the question of which one being more comfortable. 4 out of 5 consumers responded that the invention product was more comfortable while 1 out 5 consumers responded that the comparison invention product was more comfortable.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A disposable absorbent article having a longitudinal direction and a transversal direction perpendicular to the longitudinal direciton, and comprising a front region, a back region, a crotch region between the front region and the back region in the longitudinal direction, and a main body comprising an absorbent core disposed to cover continuously the crotch region and at least part of the front region and back region, wherein the front and back regions are connected each other such that a first and a second seams are formed so that the absorbent article comprises leg openings and a waist opening,
wherein the front region comprises, in order in the longitudinal direction,
a front waist zone forming a waist edge and having a front waist elastic force factor,
a front first zone having a front first zone elastic force factor,
a front second zone having a front second zone elastic force factor,
a front third zone having a front third zone elastic force factor, and
a front fourth zone having a front fourth zone elastic force factor,
wherein the front second zone includes a virtual transversal line bisecting the first and second side seams in a longitudinal direction,
wherein the front second zone elastic force factor is lower than the front first zone elastic force factor and the front third zone elastic force factor, and
wherein the front first zone elastic force factor is higher than the front third zone elastic force factor.

2. The disposable absorbent article of claim 1, wherein the front second zone has a length of about 30% to about 65% of the length of the first seam.

3. The disposable absorbent article of claim 1, wherein the front third zone elastic force factor is higher than the front fourth zone elastic force factor.

4. The disposable absorbent article of claim 1, wherein the front waist elastic force factor is higher than the front first zone elastic force factor.

5. The disposable absorbent article of claim 1, wherein the back region comprises in order in the longitudinal direction:
a back waist zone forming a waist edge and having a back waist elastic force factor,
a back first zone having a back first zone elastic force factor,
a back second zone having a back second zone elastic force factor,
a back third zone having a back third zone elastic force factor, and
a back fourth zone having a back fourth zone elastic force factor,
wherein the back second zone includes a virtual transversal line bisecting the first and second side seams in a longitudinal direction, and
wherein the back second zone elastic force factor is lower than the back first zone elastic force factor and the back third zone elastic force factor.

6. The disposable absorbent article of claim 1, wherein the front waist zone and front first zone have a length in total in a longitudinal direction of about 15% to about 35% of the length of the first seam.

7. The disposable absorbent article of claim 1, wherein the front waist zone has a length in a longitudinal direction of about 9% to about 29% of the length of the first seam.

8. The disposable absorbent article of claim 1, wherein the front third zone and the front fourth zone have a length in a longitudinal direction in the range of from about 20 mm to about 60 mm.

9. The disposable absorbent article of claim 5, wherein at least one of the front fourth zone and the back fourth zone has no stretchability.

10. The disposable absorbent article of claim 1, wherein the second zone overlaps the main chassis.

11. A disposable absorbent article having a longitudinal direction and a transversal direction perpendicular to the longitudinal direciton, and comprising a front region, a back region, a crotch region between the front region and the back region in the longitudinal direction, and a main body comprising an absorbent core disposed to cover continuously the crotch region and at least part of the front region and back region, wherein the front and back regions are connected each other such that a first and a second seams are formed so that the absorbent article comprises leg openings and a waist opening,
wherein the back region comprises, in order in the longitudinal direction,
a back waist zone forming a waist edge and having a back waist elastic force factor,
a back first zone having a back first zone elastic force factor,
a back second zone having a back second zone elastic force factor,
a back third zone having a back third zone elastic force factor, and
a back fourth zone having a back fourth zone elastic force factor,
wherein the back second zone includes a virtual transversal line bisecting the first and second side seams in a longitudinal direction, and
wherein the back second zone elastic force factor is lower than the back first zone elastic force factor and the back third zone elastic force factor.

12. The disposable absorbent article of claim 11, wherein the front first zone elastic force factor is higher than the front third zone elastic force factor.

13. The disposable absorbent article of claim 11, wherein the back second zone has a length of about 30% to about 65% of the length of the first seam.

14. The disposable absorbent article of claim 11, wherein the back second zone elastic force factor is higher than the back fourth zone elastic force factor.

15. The disposable absorbent article of claim 11, wherein the back waist elastic force factor is higher than the back first zone elastic force factor.
